(19) United States
(12) Patent Application Publication
(10) Pub. No.: US 2013/0263647 A1
(43) Pub. Date: Oct. 10, 2013

(54) APPARATUS AND METHOD FOR DETECTING GASES CONVEYED BY DRILLING FLUIDS FROM SUBTERRANEAN WELLS

(71) Applicants: Colin Lynn Barrett, Calgary (CA); Wesley Barrett, Calgary (CA)

(72) Inventors: Colin Lynn Barrett, Calgary (CA); Wesley Barrett, Calgary (CA)

(21) Appl. No.: 13/710,474

(22) Filed: Dec. 11, 2012

Related U.S. Application Data
(60) Provisional application No. 61/569,363, filed on Dec. 12, 2011.

(51) Int. Cl. G01N 33/00

(52) U.S. Cl. CPC G01N 33/0016
USPC 73/31.07

(57) ABSTRACT

This application describes apparatus and methods for detecting gas content and compositions in drilling fluids while drilling a subterranean well. More specifically, the apparatus includes an agitated drilling fluid trap incorporating an inertial bypass filter and analyzer to rapidly extract and analyze gases liberated from a drilling fluid sample. The method generally includes the steps of: extracting a gas sample from drilling fluid using an agitation system and inertial by-pass filter; analyzing the gas sample within an analyzer to obtain output signals representing gas content and composition; and, displaying the output in conjunction with other correlated drilling parameters.

### RELATED APPLICATIONS

This application is related to and claims priority from co-pending U.S. Provisional Patent Application No. 61/569,363, filed Dec. 12, 2011, the entire contents of which are fully incorporated herein by reference for all purposes.

### FIELD OF THE INVENTION

This application describes apparatus and methods for detecting gas content and compositions in drilling fluids while drilling a subterranean well. More specifically, the apparatus includes an agitated drilling fluid trap incorporating an inertial bypass filter and analyzer to rapidly extract and analyze gases liberated from a drilling fluid sample. The method generally includes the steps of: extracting a gas sample from drilling fluid using an agitation system and inertial by-pass filter; analyzing the gas sample within an analyzer to obtain output signals representing gas content and composition; and, displaying the output in conjunction with other correlated drilling parameters.

### BACKGROUND OF THE INVENTION

By way of background and with reference to FIG. 1, a conventional drilling rig 10 having a gas detector 12 is described. As is known, in conventional drilling operations drilling fluid or “mud” is pumped by a mud pump 14 down a drill string 16 to a drill bit 18. The drilling fluid passes through ports on the drill bit whereupon it is circulated back up to the surface through the borehole annulus 20 to the surface where it processed over a shaker 22 and delivered to a mud tank 24 for processing and ultimately recirculation down the well.

As is well known, the main functions of the drilling fluid are to cool and lubricate the drill bit and drill string, maintain hydrostatic pressure in the well bore to prevent fluid and gas from blowing out to the surface, carry rock cuttings to the surface and to generally clean and/or stabilize the hole as it is being drilled.

During drilling, gases and/or fluids are liberated from the formation rock which becomes entrained in the drilling fluid. As the drilling fluid is at a relatively higher pressure at the drill bit due to hydrostatic pressure forces, as the drilling fluid returns to surface, the hydrostatic pressures are released and pressurized gases will be liberated from the drilling fluid. Upon reaching the surface, the recovered solids and liquids will be also be subjected to various solids control methods and/or surface processes which further causes the release of gases from the drilling fluid.

As the composition/concentration of released gases can be highly dangerous, at surface, it is very important to quickly ascertain both the composition and concentration of the gases being released in order to ensure that all safety standards are met for both rig personnel and equipment. As well, it also very important to be able to monitor the progress of the drilling from a geological/economic perspective through knowledge of the gas composition/concentrations.

More specifically, from a safety perspective, as hydrostatic pressure is released as the drilling fluid returns to the surface, gases entrained in the drilling fluid will be liberated which depending on the composition and concentration can lead to explosive conditions and/or toxic conditions at the surface. Similarly, from an economic perspective, an indication of the composition and concentration of the gases can indicate that the drilling is entering the pay zone of a formation.

Presently, liberated gases are sampled in various sampling apparatus that are normally located at the upstream end of a shaker where the drilling fluid is initially exposed to surface pressure. Various systems of sampling have been utilized in the past with the objective to provide a timely and accurate assessment of the liberated gas.

Typically, in conventional gas detection systems as shown in FIG. 2, a motor driven drilling fluid agitation system 30 is used to rotate bars of metal, plastic or rubber within a trap 32. The trap generally consists of a closed cylinder with a fluid inlet 34 in the base and larger diameter fluid exhaust 36 and a second smaller outlet 38 for air/gas sample withdrawal.

One problem with this system is that the agitation bars will generally need to be replaced periodically due to wear and can affect gas readings as they become worn. In addition, with this agitation and trap method, fluctuating fluid levels within the delivery system will alter the consistency of gas samples as less or more fluid is drawn through the trap.

This design may also plug off the system with large fluctuations of mud in the delivery system. Still further, with larger traps, the air/gas mix can skew the absolute reading such that the reading is lower than the actual gas levels.

Still further, in these conventional gas detection systems, other problems are manifested within filtration/analyzer equipment. For example, such systems may utilize a motor driven vacuum pump with constant flow to draw air through the gas trap where a portion is transported via plastic tubing through at least one glycol bubble jar 40 and calcium chloride tablet-filled jar 42 for filtration prior to entering a gas analyzer 44. In this arrangement, the combination of the gas trap volume and glycol and calcium chloride jars increases overall volume of the sample that is analyzed and results in a diluted sample gas. Importantly, sample gas that has been diluted can also result in data that appears smoothed and/or dampened.

Further still, in past systems, the gas is then passed through gas analyzers which will typically consist of two basic types of analyzers including (1) a hotwire bridge type gas detector which has two filaments maintained at different temperatures to make it possible to distinguish between wet and dry gas; and, (2) an infra-red analyzers which can detect methane and propane along with other hydrocarbons. These analyzers must be kept at normal operating temperature ranges in order to detect and provide accurate gas readings. The gas readings can then be sent wirelessly via transmitting and receiving radios to the operator's computer located at the drilling location.

Analysis computers will typically have software that receives the incoming gas readings and adjusts them for inaccuracies in the analyzers, trap and filter systems. The gas reading data is then generally displayed on a chart, with other drilling parameters such as the rate of penetration. Common software will also typically display/process data from electronic drilling recorder systems (eg. Pason™) software to determine well bore depth, pump activity, and rate of penetration.

As is known, the display of data lags the rate of penetration data as it takes minutes to hours for drilling gas samples entrained in drilling fluid to return to the surface from the source at the drill bit location. As a result, typical software uses a manually inputted annular velocity of the drilling fluid moving along the annulus and the known depth of the well at a given time in order to match the gas sample reading to a particular depth.

In view of the foregoing, there has been a need for improved gas sampling systems and methods that overcome the foregoing problems. More specifically, there has been a need for gas detection systems and methods that improve the speed, accuracy and reliability of obtaining gas composition/concentration data from a drilling fluid. Further still, there has been a need for systems that are easy to install, require less maintenance whilst providing improved geological data.

### SUMMARY OF THE INVENTION

In accordance with the invention, there is provided a gas detection and analysis system comprising: a gas trap containing a centrifugal pump and agitation system for operative connection to a drilling fluid source; and an inertial bypass filter operatively connected to the gas trap for receiving a gas flow sample liberated from within the gas trap, the inertial bypass filter having a sample port for withdrawing a gas sample from the bypass filter.

In one embodiment, the system includes a gas analyzer operatively connected to the sample port for receiving and analyzing the gas sample from the bypass filter.

In further embodiments, the gas trap includes a gas trap chamber having a fluid outflow port for discharging drilling fluid from the gas trap and establishing a maximum drilling fluid height within the gas trap. The gas trap preferably includes a gas outflow port above the fluid outflow port for discharging the gas flow sample to the inertial bypass filter.

In one embodiment, the inertial bypass filter has a bypass filter outflow port operatively connected to the gas trap for returning gas flow sample to the gas trap. Preferably, the inertial bypass filter has a tube body substantially parallel to gas flow and the sample port is substantially perpendicular to the gas flow and the tube body includes a filter medium on the interior surface of the tube body.

In other embodiments, the centrifugal pump and agitation system includes a right-handed or left-handed auger extending through the gas trap chamber wherein rotation of the auger controls drilling fluid intake and liberation of gas from drilling fluid. Preferably, the auger is rotated in a direction to promote the downward flow of gas/drilling fluid within the gas trap chamber.

In another embodiment, the gas trap chamber has a smaller diameter at the top relative to the outer diameter of the auger wherein rotation of the auger within the gas trap chamber creates a vacuum at the top of the gas trap chamber to draw the gas flow sample from the gas trap to the inertial bypass chamber and back to the gas trap chamber.

In other embodiment, the system also includes a temperature compensated mass flow meter operatively connected to the inertial bypass filter and analyzer for maintaining sufficient sample flow to the analyzer.

In a more specific embodiment, the invention provides gas detection and analysis system comprising: a gas trap containing an auger for operative connection to a drilling fluid source, the gas trap having a gas trap chamber having: a fluid outflow port for discharging drilling fluid from the gas trap and establishing a maximum drilling fluid height within the gas trap; and, a gas outflow port above the fluid outflow port for discharging a gas flow sample to an inertial bypass filter; the inertial bypass filter operatively connected to the gas trap for receiving the gas flow sample liberated from within the gas trap, the inertial bypass filter having: a tube body substantially parallel to gas flow and wherein the sample port is substantially perpendicular to the gas flow; a bypass filter outflow port operatively connected to the gas trap for returning gas to the gas trap; a gas analyzer operatively connected to the sample port for receiving and analyzing the gas sample from the bypass filter; wherein the gas trap chamber has a smaller diameter at the top relative to the outer diameter of the auger and wherein rotation of the auger within gas trap chamber creates a vacuum at the top of the gas trap chamber to draw the gas flow sample from the gas trap to the inertial bypass chamber and back to the gas trap chamber.

In another aspect, the invention provides a method for sampling and analyzing gas concentration and composition within a drilling fluid comprising the steps of: agitating a drilling fluid sample within a gas trap to liberate entrained gas from the drilling fluid sample; passing the entrained gas through an inertial bypass filter operatively connected to the gas trap; and withdrawing a gas sample from the bypass filter.

In another embodiment, the method includes the step of analyzing the gas sample from the bypass filter and/or drawing the sample through the inertial bypass filter by a vacuum created at the top of the gas trap.

In another embodiment, the method includes the step of withdrawing the gas sample at an angle substantially perpendicular to the flow of gas through the inertial bypass filter.

### BRIEF DESCRIPTION OF THE DRAWINGS

The invention is described with reference to the Figures, in which:

FIG. 1 is a schematic diagram of a typical drilling rig in accordance with the prior art;

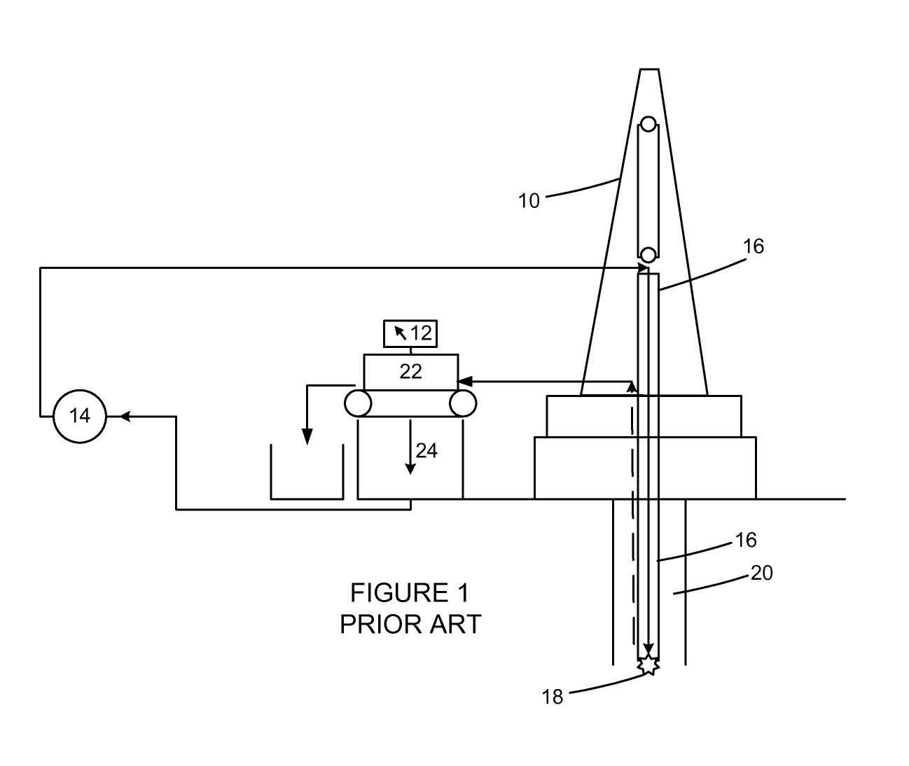

FIG. 2 is a schematic diagram of typical gas detection system in accordance with the prior art;

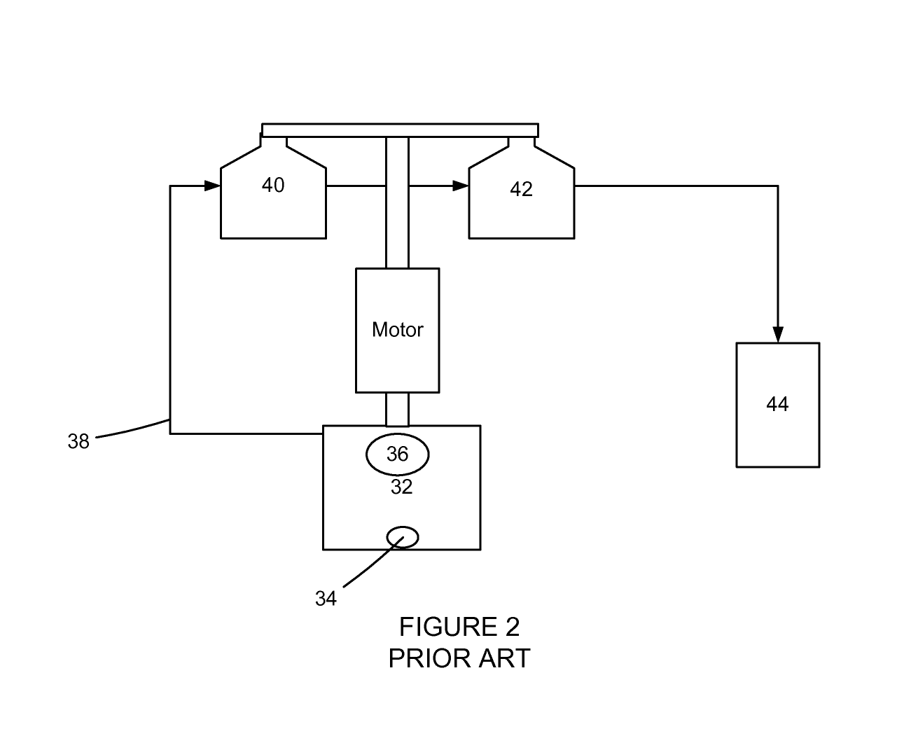

FIG. 3 is a schematic diagram of a gas detection system in accordance with one embodiment of the invention;

FIG. 4 is a schematic diagram of a bypass filter in accordance with one embodiment of the invention; and,

FIG. 5 is a schematic diagram of a communication system in accordance with one embodiment of the invention.

### DETAILED DESCRIPTION OF THE INVENTION

With reference to the Figures, improved gas detection and analysis systems and methods of operation are described.

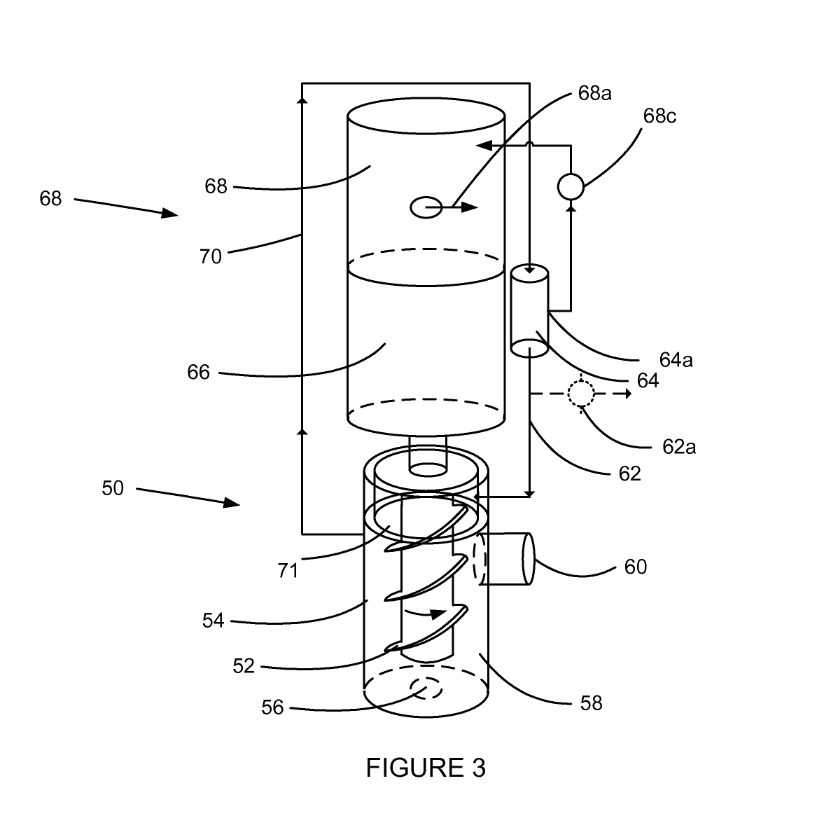

With reference to FIG. 3, a gas detector 50 enabling improved gas concentration and composition measurements is described. The gas detector generally includes a low volume centrifugal auger/bit 52 within a trap 54, drilling fluid intake 56, gas/fluid exhaust 60, filter waste exhaust/trap vacuum line 62, inertial bypass filter 64, motor 66 and gas analyzer 68.

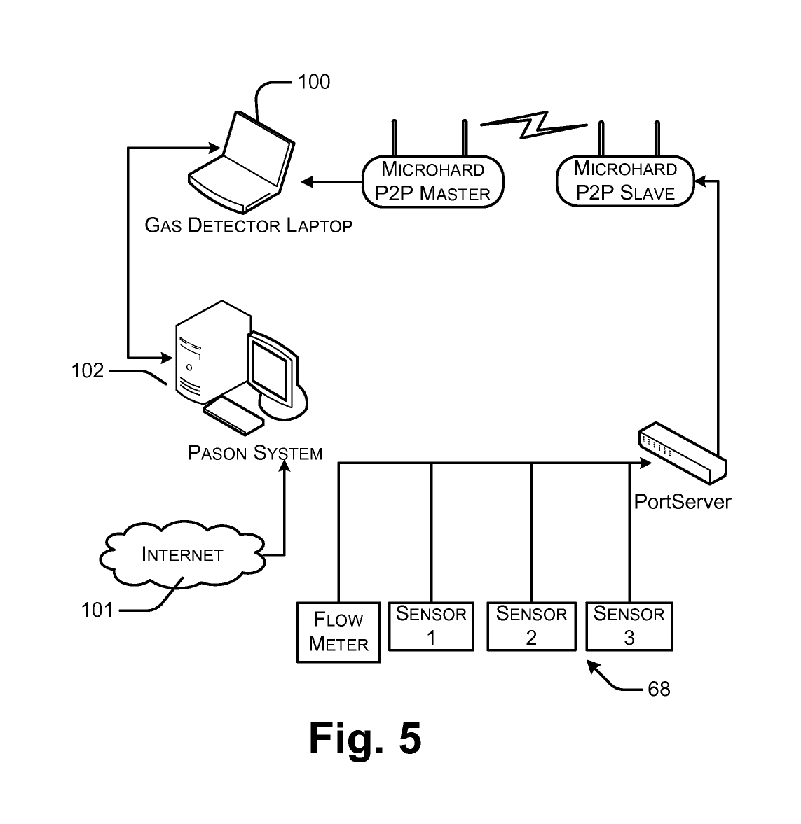

Preferably, the analyzer is a digital, temperature compensated, infrared radiation analyzer operable to efficiently determine methane, propane hydrocarbon content, carbon dioxide, and hydrogen sulphide concentrations from within a drilling fluid. In addition, the system also includes radio communication devices, computer and software as shown in FIG. 5.

In operation, the gas trap/drilling fluid intake 56 is positioned within drilling fluid as it exits the well such that drilling fluid is allowed to enter the trap 54. Normally, this location will be in the possum belly tank (header tank) of a shaker. With the auger rotating, mechanical agitation causes the release of entrained gases within the trap. That is, as the auger rotates, the fluid and gas mix is agitated/centrifuged whereby the fluid is propelled toward and around the interior walls of the trap housing, and subsequently out the trap fluid exhaust 60, whilst liberated gas is drawn through standpipe 70 and bypass filter 64 before exiting through waste line/vacuum line 62 and subsequently out of the system. A representative gas sample is drawn from the inertial bypass filter for analysis within the gas analyzer 66 and removal from the system through port 68a. Preferably, the auger (or equivalent) is rotated in a counterclockwise direction (assuming a right handed auger) in order to control the flow of drilling fluid into the trap through the intake.

In one embodiment, as shown in FIG. 3, vacuum/draw created from the auger bit and a reduced inside diameter 71 at the trap top is also used to draw the gas sample through the bypass filter 64. In this case, waste line 62 from the bypass filter 64 is coupled to the trap top, wherein waste gas from the bypass filter is drawn into the top of the trap. This creates a simple, closed system such that sample gas cannot be introduced to the inertial bypass filter 64 via the waste/exhaust port 60 of the bypass filter 64. Importantly, this layout generally increases the speed in which the gas sample gets to the analyzer, and increases filter efficiency. In other embodiments, a separate vacuum/pump/valve system 62a could be utilized to draw waste gas through the bypass filter and away from the system.

In addition, the low volume trap and reverse (counterclockwise rotation) centrifugal auger mud agitator provides a substantially constant volumetric fluid flow through the trap for the liberation of gases from the drilling fluid with the result being that a small representative gas sample is drawn up the standpipe 70.

In order to overcome the problems of past systems, the auger bit is preferably constructed of hardened and tempered steel and/or other harder materials to reduce wear from abrasive cuttings. The auger bit can be of any suitable diameter relative to the inside diameter of the trap to effectively allow for the agitation of the drilling fluid to release the entrained gases and allow for an appropriate flow of liquids and gases through the system. Generally, a smaller sized trap is more beneficial to reduce overall gas trap volume, and dilution of liberated gas.

Importantly, this design also reduces/eliminates trap flooding and the resultant sample line plugging by controlling mud intake to the trap.

### Bypass Filter

From the standpipe 70, the air/gas sample enters the inertial bypass filter 64 where solid contaminants and fluid are separated from the air/gas sample. The inertial bypass filter is a tube 64d having an axis generally parallel to gas flow. The use of a bypass filter system reduces associated maintenance time and costs as the need to replace disposable filters that would otherwise plug off and affect data accuracy as is common in the prior art is significantly reduced. Importantly, unlike conventional units which can take 1 to 5 minutes to read the gas levels in the drilling mud, the use of the subject bypass filter systems allows gas sampling times to be less than 30 seconds.

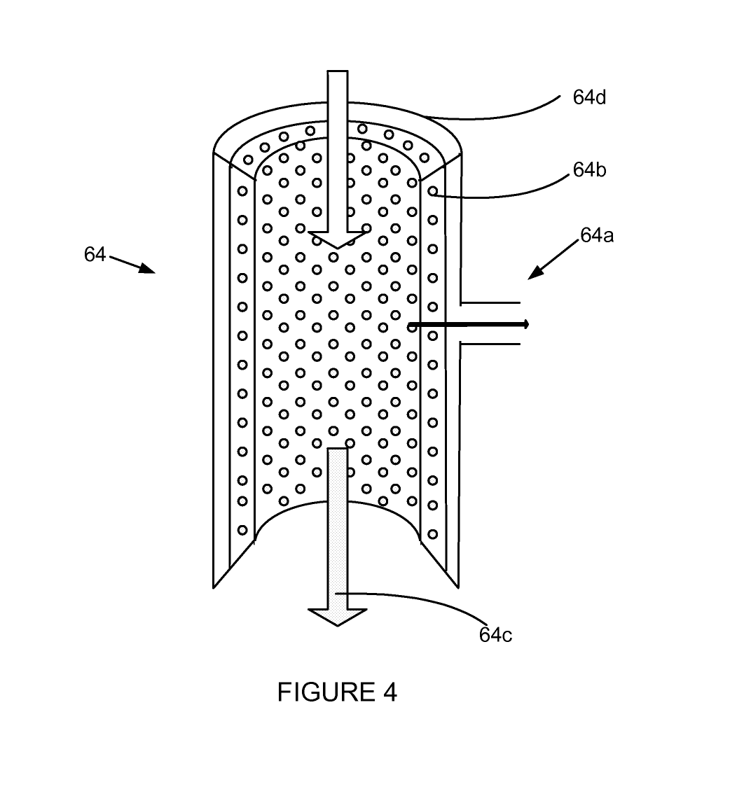

That is, in the subject system, relatively larger volumes of particulate laden gas pass straight through the filter element as shown in FIG. 4 wherein the gas sample to be analyzed is drawn from a bypass flow through a port 64a which is perpendicular to the main flow. As the sample is pulled off as a bypass stream, the sample is filtered by the filter materials 64b whereas the bulk of the particulate 64c passes directly through the length of the filter element due to its velocity and inertia created from the gas trap vacuum, which prevents particulate from being diverted by the pull of the sample outlet port. Any particles that are pulled to the sample outlet are filtered by the filter material thus providing a filtered sample for analysis. Subsequent combinations of filters may also be used in combination to further clean the sample.

In addition, the flow velocity also continuously flushes the surface of the filter element, thereby providing effective self cleaning which reduces pore clogging.

Generally, a typical analyzer will only draw a sample flow of 1-2 liters per minute through a large volume sample conditioning system. As such, the time required to pull the sample from the sample point to the analyzer is reduced by the bypass filter, thus providing a more accurate time correlation to the gas sample entering the analyzer relative to the composition of the drilling fluid entering the trap. The sample is less diluted in the small volume system, which is of particular importance under high rate of penetration drilling conditions.

### Analyzer and Sensors

Within the analyzer, infrared analyzers determine the, methane, propane, carbon dioxide, and hydrogen sulphide content as well as the presence/concentrations of other hydrocarbons that can be used by the operators to determine geological information about the formation.

In a preferred embodiment, the sensors within the analyzer are fully temperature-compensated sensors that provide very reliable readings, and have digital output for direct communication with instrument electronics (eg. Dynament Ltd., South Normanton, Derbyshire, United Kingdom). The infrared sensors operate using nondispersive infrared sensor (or NDIR) principles to monitor the presence of the target gas. The sensors contain long-life tungsten filament infrared light sources, an optical cavity into which gas diffuses, a dual temperature compensated pyroelectric infrared detector, an integral semiconductor temperature sensor and electronics to process the signals from the pyroelectric detector. The digital output is a UART format comprising 8 data bits, 1 stop bit and no parity.

High resolution infrared IR sensor measures methane from 0 to 100% volume with resolution of 0.01% for 0-10% methane and 0.1% for 10-100% volume therefore enabling the accurate measurement of 0-100% volume methane with one sensor. Each sensor contains all the necessary optics, electronics and firmware to provide a linearised, temperature-compensated output. Operating temperature ranges of −20° C. to +50° C. is sufficient with the heat produced from the other electrical components in the analyzer housing. Other hydrocarbons that the methane sensor is cross sensitive to include ethane, propane, butane, pentane, hexane, ethylene, ethanol, propylene, and cyclopentane.

The infrared propane sensor includes a range of miniature infrared IR sensors for the measurement of propane gas (Dynament Ltd.) The sensor is fully characterized for the detection of propane gas over the range 0-2% volume. Other hydrocarbons that the propane sensor is cross sensitive to in lesser amounts include butane, pentane, hexane, ethanol, ethylene, propylene, ethane, cyclopentane, isopropanol, methanol, toluene, acetone, methyl ethyl ketone, (MEK) and xylene. The propane sensor will generally read higher quantities of heavier hydrocarbons than the methane sensor.

The carbon dioxide infra red sensor (Dynament Ltd.) has a measurement range 0-500 ppm up to 0-5% Volume and 0-100% volume CO₂.

All 3 sensors have comparable temperature compensated output.

### Flow Meter

The system also preferably includes a temperature compensated mass flow meter 68c (Alicat M Series, Alicat Scientific Inc. Tuscon, Ariz.) in the analyzer housing to ensure sufficient sample flow is being analyzed, and that there are no obstructions. Unlike other units using rotameters, temperature compensated mass flow meters address the problems associated with other units that often seize.

### Communications

As shown in FIG. 5, in one embodiment, the system includes an appropriate network interface to enable the system to wirelessly communicate all readings to a logging laptop computer 100 at the wellsite. In addition, the system may be connected to other rig control systems 101 as well as the internet 102. In one embodiment, the sensors can be re-programmed and/or calibrated as well over the wireless connection. Reprogramming or diagnosing problems can also be remotely controlled. The wireless communication is encrypted and utilizes frequency hopping to alleviate loss of connection frequently a problem with some other units currently in use.

### Other Design Features

In a preferred embodiment, the system is pre-assembled and is a compact size to simplify installation. Preferably, the operator can simply mount the unit to the drilling rig equipment using a quick clamp mount and plug the system in.

Reading resolution is also increased due to the decrease in air volume of the sampling setup, which includes the trap, and filter system. This enables an operator/geologist to better understand exactly what zone is producing the gas response.

Maintenance is also reduced as the bypass filtration system requires less operator involvement, and no use of glycol or dryer agents.

Freeze-up of sample lines is also reduced as sample lines are short and are kept relatively warmer subsequent to exiting the standpipe causing less condensation/buildup in the lines.

Although the present invention has been described and illustrated with respect to preferred embodiments and preferred uses thereof, it is not to be so limited since modifications and changes can be made therein which are within the full, intended scope of the invention as understood by those skilled in the art.

1. A gas detection and analysis system comprising:
a gas trap containing a centrifugal pump and agitation system for operative connection to a drilling fluid source;
an inertial bypass filter operatively connected to the gas trap for receiving a gas flow sample liberated from within the gas trap, the inertial bypass filter having a sample port for withdrawing a gas sample from the bypass filter.

2. The gas detection and analysis system as in claim 1 further comprising a gas analyzer operatively connected to the sample port for receiving and analyzing the gas sample from the bypass filter.

3. The gas detection and analysis system as in claim 1 wherein the gas trap includes a gas trap chamber having a fluid outflow port for discharging drilling fluid from the gas trap and establishing a maximum drilling fluid height within the gas trap.

4. The gas detection and analysis system as in claim 3 wherein the gas trap includes a gas outflow port above the fluid outflow port for discharging the gas flow sample to the inertial bypass filter.

5. The gas detection and analysis system as in claim 1 wherein the inertial bypass filter has a bypass filter outflow port operatively connected to the gas trap for returning gas flow sample to the gas trap.

6. The gas detection and analysis system as in claim 1 wherein the inertial bypass filter has a tube body substantially parallel to gas flow and the sample port is substantially perpendicular to the gas flow.

7. The gas detection and analysis system as in claim 6 wherein the tube body includes a filter medium on the interior surface of the tube body.

8. The gas detection and analysis system as in claim 3 wherein the centrifugal pump and agitation system includes a right-handed or left-handed auger extending through the gas trap chamber wherein rotation of the auger controls drilling fluid intake and liberation of gas from drilling fluid.

9. The gas detection and analysis system as in claim 8 wherein the auger is rotated in a direction to promote the downward flow of gas/drilling fluid within the gas trap chamber.

10. The gas detection and analysis system as in claim 5 wherein the gas trap chamber has a smaller diameter at the top relative to the outer diameter of the auger wherein rotation of the auger within gas trap chamber creates a vacuum at the top of the gas trap chamber to draw the gas flow sample from the gas trap to the inertial bypass chamber and back to the gas trap chamber.

11. The gas detection and analysis system as in claim 2 further comprising a network interface operatively connected to the analyzer for communication of sensor data to a central computer.

12. The gas detection and analysis system as in claim 2 further comprising a temperature compensated mass flow meter operatively connected to the inertial bypass filter and analyzer for maintaining sufficient sample flow to the analyzer.

13. A gas detection and analysis system comprising:
a gas trap containing an auger for operative connection to a drilling fluid source, the gas trap having a gas trap chamber having:
a fluid outflow port for discharging drilling fluid from the gas trap and establishing a maximum drilling fluid height within the gas trap; and,
a gas outflow port above the fluid outflow port for discharging a gas flow sample to an inertial bypass filter;
the inertial bypass filter operatively connected to the gas trap for receiving the gas flow sample liberated from within the gas trap, the inertial bypass filter having:
a tube body substantially parallel to gas flow and wherein the sample port is substantially perpendicular to the gas flow;
a bypass filter outflow port operatively connected to the gas trap for returning gas to the gas trap;
a gas analyzer operatively connected to the sample port for receiving and analyzing the gas sample from the bypass filter;
wherein the gas trap chamber has a smaller diameter at the top relative to the outer diameter of the auger and wherein rotation of the auger within gas trap chamber creates a vacuum at the top of the gas trap chamber to draw the gas flow sample from the gas trap to the inertial bypass chamber and back to the gas trap chamber.

14. A method for sampling and analyzing gas concentration and composition within a drilling fluid comprising the steps of:
agitating a drilling fluid sample within a gas trap to liberate entrained gas from the drilling fluid sample;
passing the entrained gas through an inertial bypass filter operatively connected to the gas trap; and
withdrawing a gas sample from the bypass filter.

15. A method as in claim 14 further comprising the step of analyzing the gas sample from the bypass filter.

16. A method as in claim 14 further comprising the step of drawing the sample through the inertial bypass filter by a vacuum created at the top of the gas trap.

17. A method as in claim 14 further comprising the step of withdrawing the gas sample at an angle substantially perpendicular to the flow of gas through the inertial bypass filter.
